(19) United States
(12) Patent Application Publication
(10) Pub. No.: US 2008/0294254 A1
(43) Pub. Date: Nov. 27, 2008

(54) Intraocular lens

(76) Inventors: J. Stuart Cumming, Laguna Beach, CA (US); Jonathan R. Soiseth, Pomona, CA (US); Michael J. Breen, Mission Viejo, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/974,364

(22) Filed: Oct. 11, 2007

Related U.S. Application Data
(63) Continuation-in-part of application No. 11/549,818, filed on Oct. 16, 2006.
Application No. 11/549,818 is a division of application No. 11/295,924, filed on Dec. 6, 2005, now abandoned.

(51) Int. Cl. A61F 2/16; B29D 11/00

(52) U.S. Cl. 623/6.37; 264/2.7

(57) ABSTRACT

An accommodating intraocular lens where the optic is moveable relative to the outer ends of the extended portions. The lens comprises an optic made from a flexible material combined with extended portions that are capable of multiple flexions without breaking. The optic has a blended central area of increased power of 1 diopter or less to give the patient a single focal point on wavefront examination after implantation into the eye increasing the depth of focus to aid near vision.

### CROSS REFERENCE TO RELATED APPLICATIONS

This application is a continuation-in-part of application Ser. No. 11/459,818 filed Oct. 16, 2006, which is a divisional of application Ser. No. 11/295,924 filed on Dec. 6, 2005, now abandoned, which are expressly incorporated herein by reference.

### BACKGROUND

Intraocular lenses have for many years had a design of a single optic with loops attached to the optic to center the lens and fixate it in the empty capsular bag of the human lens. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990s an acrylic material was introduced as the optic of lenses. The acrylic lens comprised a biconvex optic with a straight edge into which were inserted loops to center the lens in the eye and fixate it within the capsular bag.

Flexible acrylic material has gained significant popularity among ophthalmic surgeons. In 2003 more than 50% of the intraocular lenses implanted had acrylic optics. Hydrogel lenses have also been introduced.

The advent of an accommodating lens which functions by moving along the axis of the eye by repeated flexions somewhat limited the materials from which the lens could be made. Silicone is a suitable material, since it is flexible and can be bent probably several million times without showing any damage. Additionally a groove or hinge can be placed across the plate adjacent to the optic as part of the lens design to facilitate movement of the optic relative to the outer ends of the haptics. On the other hand, some acrylic materials fracture if repeatedly flexed.

Recently accommodative or accommodating intraocular lenses have been introduced to the market, which generally are modified plate haptic lenses and, like the silicone plate haptic lenses, the first accommodating lenses had no clear demarcation between the junction of the plate with the optic's posterior surface. A plate haptic lens may be referred to as an intraocular lens having two or more plate haptics joined to the optic. The latest plate haptic accommodating lens has a square edge on the posterior side of the optic.

### SUMMARY OF THE INVENTION

According to a preferred embodiment of this invention, an accommodating lens comprises a lens with a flexible solid optic attached to which are two or more extended portions which may be plate or loop haptics capable of multiple flexions without breaking, preferably along with fixation and centration features at their distal ends. There may be a hinge or groove across the extended portions adjacent to the optic to facilitate the anterior and posterior movement of the optic relative to the outer ends of the extended portions. The extended portions are preferably plate haptics which may have parallel sides or be narrower or wider adjacent to the optic.

The center of the optic of the lens of the present invention has a central area of 1.0 diopter or less with a diameter of 1.0 to 2.5 mm preferably on the front surface to aid in near vision. After the lens is implanted into the eye of a patient wavefront analysis demonstrates a single focal point on the retina of the patient. Patients do not complain of glare or halos as they do with standard multifocal lenses.

After the lens is manufactured, it is tumbled with a slurry of glass beads to remove any flashing, smooth the edges and integrate the radii. Before tumbling, the central power radius on an optical pin is designed to give the optic a central power 1.5 diopters more than the power in the periphery of the lens. After tumbling the power of the central area was found to be reduced to 1.0 diopter or less. The lens shrank resulting in an absence of discrete radii SR1-SR5, and thus ends up not a multiple power lens after implantation into the eye. The resulting blended design after completion does not cause separate images on wavefront analysis after implantation into an eye, as does a multifocal lens, but actually provides a central defocus curve which provides additional focusing power and actually results in an extended region of depth of field about the far point of the patient's vision. Thus, a desired depth of field increase about the near focal point occurs, and the retinal image range has been determined to be superior than that of a standard accommodating intraocular lens. The through focus wavefront aberrations peak to valley and RMS graphs and waveforms described later show quantitatively how the present lens provides superior overall optical performance in the range of object vergence from infinity to 2 D. Thus, the lens functions by extending the range of accommodation about the far point by increasing the static depth of field. A patient's near vision is improved by both accommodation of the lens by axial movement, arching of the optic and by virtue of an increased depth of field. Additionally, non-accommodating lenses can be improved in the same manner.

Thus, the present invention is directed to a useful intraocular lens with an increased power in the center of the optic, the lens involving a single focal point on wavefront analysis after implantation in the eye.

Accordingly, features of the present invention are to provide an improved form of lens including a central area of increased power to improve the patient's near vision by increasing the depth of focus with an accommodating lens to give the patient a single focal point without the significant glare or halos associated with the standard multifocal lenses.

### BRIEF DESCRIPTION OF THE DRAWINGS

FIGS. 1a and 1b are perspective views of a preferred embodiment of the present invention.

FIGS. 2a and 2b are front elevational views.

FIG. 3 is a side elevational view

FIG. 4 is an end view.

FIG. 5 illustrates the lens, showing T-shaped haptics engaged in the capsular bag having been depressed by the bag wall toward the optic.

FIGS. 6a and 6b provide details of the blended design transition of the anterior optic surface from the outside to the center of the lens.

FIGS. 6a and 6b provide details of the blended design transition of the anterior optic surface from the outside to the center of the lens.

FIGS. 7a-7e illustrate optical pin design used in the manufacture of the present lens.

According to the present invention the lens is of a foldable, flexible silicone, acrylic, collamer or hydrogel material and the haptic plates are of a foldable material that will withstand multiple foldings without damage, e.g., silicone, acrylic, collamer or hydrogel. Preferably, the end of the plate haptics have T-shaped fixation devices and are hinged to the optic

### DESCRIPTION OF THE PREFERRED EMBODIMENT

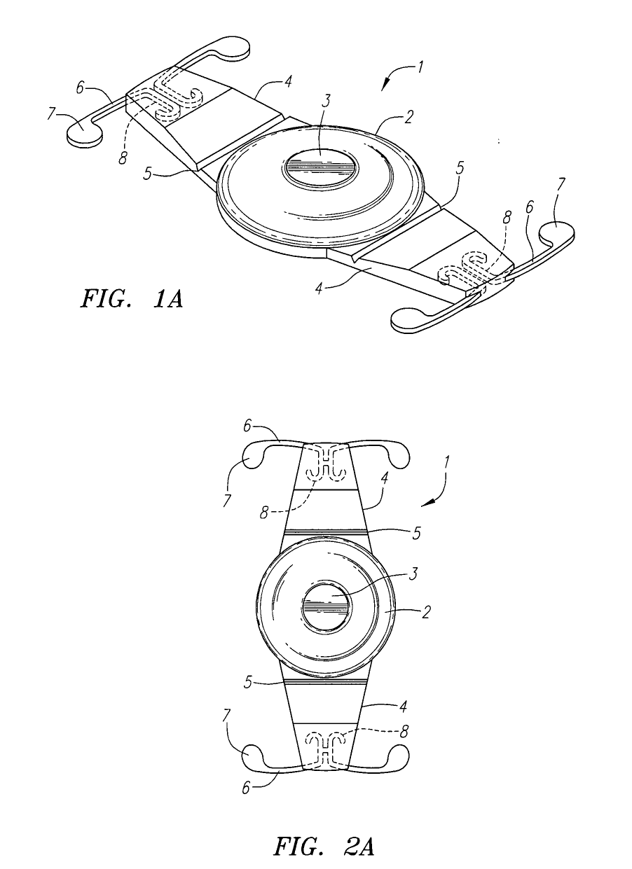

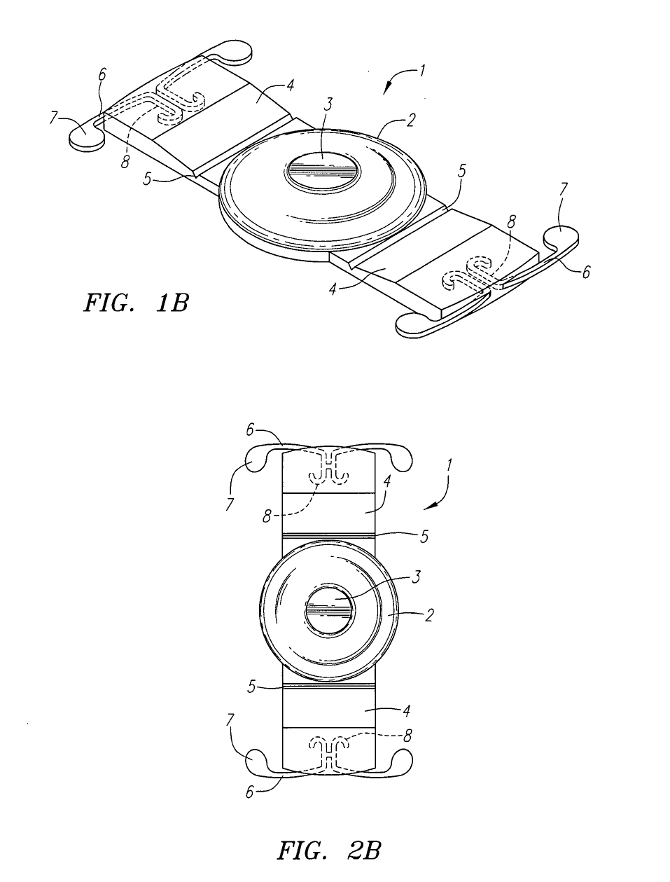

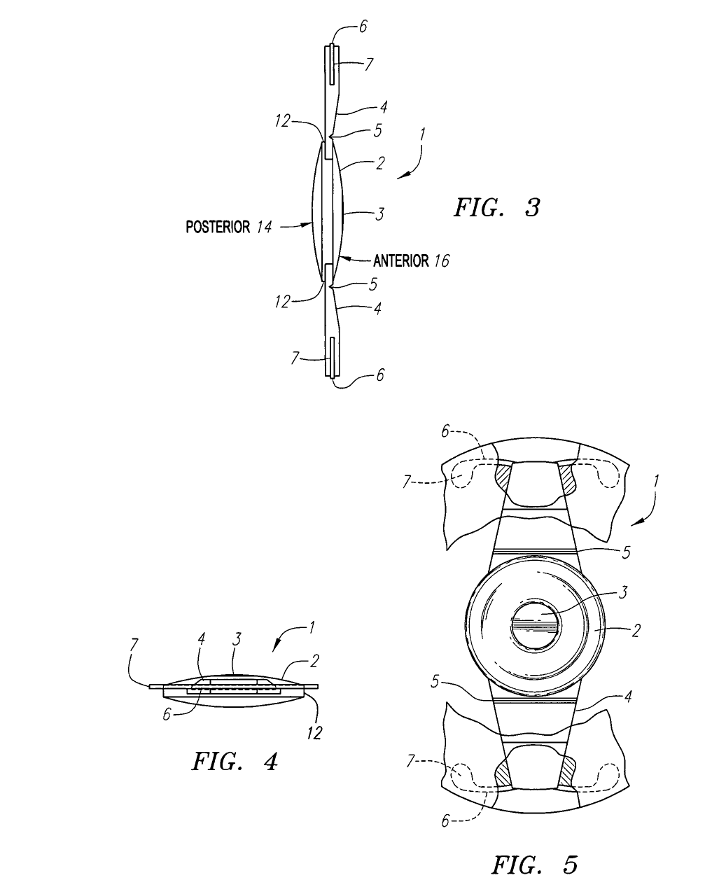

Turning now to the Figures, preferred embodiments are illustrated in detail comprising an intraocular lens 1 formed as a flexible solid optic 2 and can be made of silicone, and flexible extending portions 4 of any suitable form which may be plate haptics, open or closed loops, or fingers which are capable of multiple flexations without damage and formed, for example, of silicone, acrylic or collamer. The optic 2 and haptics 4 preferably are uniplanar, and one or more haptics 4 extend distally from sides of the optic 2. The ends of the plate haptics may have fixation and/or centration extensions which can be flexible loops or protuberances on one or both sides and/or on the edges of the plates. The plate haptics may have a square edged ridge across the posterior surface width of the plate to reduce posterior capsule opacification. The haptics can be tapered as seen in FIGS. 1a and 2a, or with parallel sides as in FIGS. 1b and 2b.

According to the present invention, the optic 2 has a central blended area 3. The lens 1 preferably comprises an accommodating intraocular lens currently available from eyeonics, inc., Aliso Viejo, Calif., such as shown in U.S. Pat. No. 6,387,126, typically with a 4.5-5.5 mm diameter optic, but with a 1.0 to 2.5 mm diameter central area 3 and which has an add of 1 diopter or less in the center of the optic 1. The area 3 can be on the anterior or posterior side of the lens, and the other side can be any conventional form or can be toric if desired, or just the posterior surface behind the bulls eye could be toric. The added power area 3 is to aid in near vision. The optic diameter can range from approximately 3.5 to 8.0 mm but a typical one is 4.5-5.5 mm. The lens optic may be biconvex, piano convex or have a Fresnell surface.

Non-accommodating intraocular lenses have been disclosed with a central area with a power of 2.0 diopters or more. Examples are in Nielson, U.S. Pat. No. 4,636,211, and Keats, U.S. Pat. No. 5,366,500. Such lenses result in the patient having two separate images, and the brain has to adapt to ignore the unwanted images.

Importantly, with the present lens, accommodating or not, having a central area of 1 diopter or less the vision appreciated by the patient will not have separate images, but the near vision will be improved through an increased depth of field.

The haptics preferably are plate haptics and preferably may be flat or curved having arcuate outer edges including loops 6. The loops 6 when unrestrained are somewhat less curved in configuration as shown in FIGS. 1-2, but flex centrally to conform to the inner diameter of the capsular bag after insertion. Compare an example of an inserted lens 1 as seen in FIG. 5. The lens 1, including the optic 2, haptics 4, and loops 6 are preferably formed of a semi-rigid material such as silicone, collamer, acrylic, or hydrogel, and particularly a material that does not fracture with time. The loops 6 can be of a material different from the haptics 4 and retained in the haptics by loops 8 molded into the ends of the haptics. Grooves or thin areas 5 forming hinges preferably extend across the haptics 4 adjacent to the optic 2. The hinges may have a wide base such that the base can stretch like an elastic band upon a posterior increase in pressure. The lens of FIG. 5 alternatively have parallel sides like in FIGS. 1b and 2b.

The flexible haptics 4 and loops 6 can be connected to an acrylic optic 2 by means of an encircling elastic band (not shown) which fits into a groove in the acrylic optic 2 as shown and described in co-pending application Ser. No. 10/888,536 filed Jul. 8, 2004 and assigned to the assignee of the present application.

There can be a sharp edge 12 around the posterior surface 14 of the optic 2. It is designed to reduce the migration of cells across the posterior capsule of the lens post-operatively and thereby reduce the incidence of posterior capsular opacification and the necessity of YAG posterior capsulotomy.

FIGS. 1a and 1b illustrate the haptics 4, loops 6, and hinge 5 across the haptics adjacent to the optic 2. Knobs 7 can be provided on the ends of the loops 6 and are designed to fixate the loops 6 in the capsular bag of the eye and at the same time allow the loops 6 to stretch along their length as the optic 2 of the lens 1 moves backward and forward and the haptics 4 move or slide within pockets formed between the fusion of the anterior and posterior capsules of the capsular bag.

The end of the loops 6 containing the knobs 7 may be either integrally formed from the same material as the haptics 4 or the loops may be of a separate material such as polyimide, prolene, or PMMA as discussed below. The loops if formed of a separate material are molded into the terminal portions of the plate haptics 4. The material of flexible loop 6 can extend by elasticity along the internal fixation member of the loop.

As noted above, the haptics 4 may have a groove or thin area 5 forming a hinge across their surface adjacent to the optic. This facilitates movement of the optic anteriorly and posteriorly relative to the outer ends of the haptics. The hinge may have a wide base allowing it to stretch like an elastic band to further allow the optic to move forward.

The present concepts are applicable to several forms of lenses, such as lenses shown in Cumming U.S. Pat. Nos. 5,476,514, 6,051,024, 6,193,750, and 6,387,126, and non-accommodating intraocular lenses also.

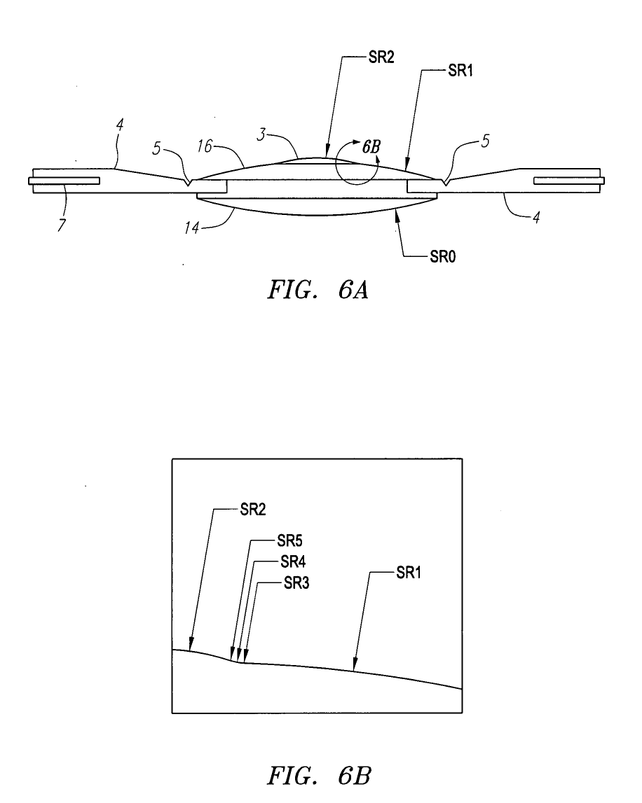

FIGS. 6a and 6b illustrate more detail of the blended design of the anterior optic surface 16 and thus show the transition of the anterior optic surface from the outside surface of spherical radius SR1 to the center surface of the spherical radius of SR2 which comprises the central area 3 illustrated in the other Figures. FIGS. 6a and 6b demonstrate the transition area as a varying radius that ranges from SR1 to SR2, and it should be noted that the difference between SR1 and SR2 has been enhanced to better show the transition. In particular, SR1 is >SR3>SR4>SR5>SR2.

As is well known in the art, the intraocular lens 1 such as that in the drawings is implanted in the capsular bag of the eye after removal of the natural lens. The lens is inserted into the capsular bag by a generally circular opening cut in the anterior capsular bag of the human lens and through a small opening in the cornea or sclera. The outer ends of the haptics 4, or loops 6, are positioned in the cul-de-sac of the capsular bag. The outer ends of the haptics, or the loops, are in close proximity with the bag cul-de-sac, and in the case of any form of loops, such as 6, the loops are deflected from the configuration as shown for example in FIG. 2 to the position shown in FIG. 5. The knobs 7 can be provided on the outer end portions of the loops 6 for improved securement in the capsular bag or cul-de-sac by engagement with fibrosis, which develops in the capsular bag following the surgical removal of the central portion of the anterior capsular bag. The present lens is intended to give superior instant near vision without patient multifocality or glare. The non-dominant eye may be implanted with the lens and the dominant eye with a lens without the central area 3 or with a similar lens to the one of this invention. The lenses are implanted in the same manner as described above and as known in the art.

There are two descriptions of the lens that should be considered.
The first is the distribution of the lens power range of 4.0 to 33.0 diopters. The most commonly used dioptic power of the lens is 22.0 diopter.
A histogram of the lens is basically a bell curve with a peak at 22.0 diopter. Often analysis is done with a 22 diopter lens for this very reason.

The second is relative to the lens design with the central portion 3 of the lens being typically 1.5 mm in diameter. The power of this area will be 1.0 diopter or less than that of the surrounding area, after tumbling as described earlier. This gives the patient a single focal point, which can be demonstrated by wavefront analysis which is the essence of this invention.

The lens design can be based on the existing eyeonics crystalens to the extent of the following:
The lens and plate haptics are manufactured from the same mold; however, one of the pins for molding the anterior optical surface of the present crystalens 5-0 has a central area of 1.5 diopter, and preferably a diameter of 1.5 mm.
Lens and plate material is Biosil (Silicone).
The plate haptics are preferably the same design as the crystalens 5-0, but can have parallel sides.
The loops are made from the same Kapton HN (polyimide).
The posterior surface radius may be the same as or different than the anterior outer radius (e.g. a 23 diopter pin on the anterior side and a 21 diopter pin on the posterior side will give a 22 diopter).

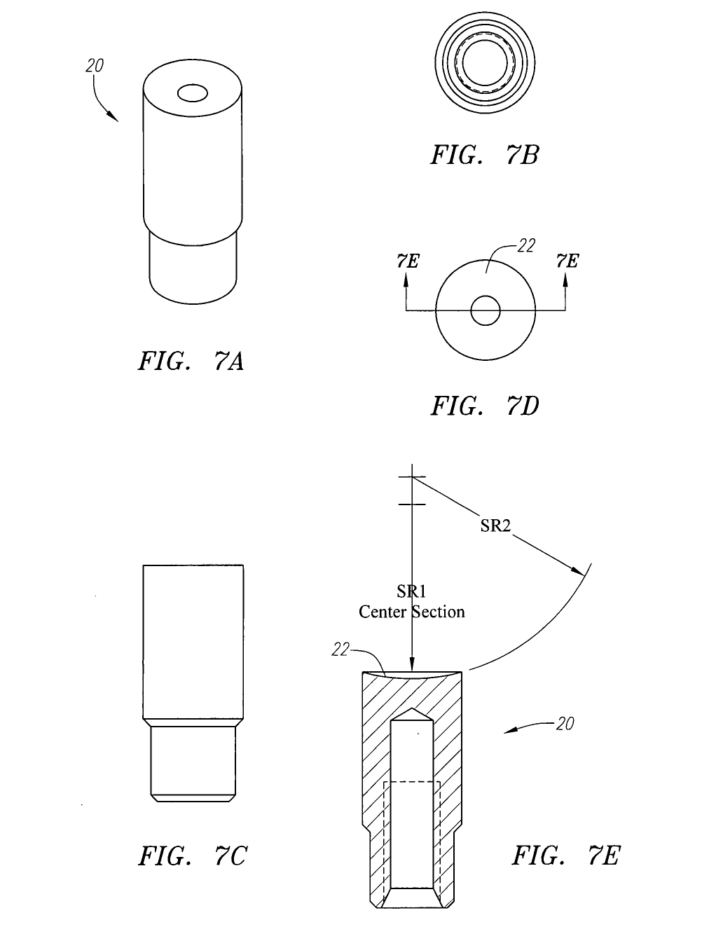

FIG. 7 illustrates the optical pin design previously discussed. FIG. 7a is a perspective view, FIG. 7b is a bottom view, FIG. 7c is a side view, FIG. 7d is an end view with the two spherical radii machined into the surface used for molding, and FIG. 7e is a cross-sectional view taken along the lines a-a of FIG. 7d. SR-1 and SR-2 represent the two radii.

Below are calculated dimensions of the optical section of the IOL for the minimum, average and maximum diopter lens. Diopter 1 is the dioptric power through the anterior outer perimeter of the lens, and Diopter 2 is through the center section. Note that the radii are approximate as SR0 (posterior surface spherical radius) and SR1 (outer anterior surface spherical radius) aren't necessarily the same. The center thickness on the center area 3 of the added power is approximately 3 microns (0.003 mm) thicker over the 4 to 33 diopter range.

| | | SR0 & | | Center |
|---|---|---|---|---|
| Diopter 1 | Diopter 2 | SR1 (mm) | SR2 (mm) | Thickness (mm) |
| 4 | 5 | 45.47 | 30.30 | 0.46 |
| 22 | 23 | 8.24 | 7.55 | 0.97 |
| 33 | 34 | 5.47 | 5.16 | 1.32 |

Returning to manufacture of the lens, the optical pin 20 design is shown in FIGS. 7a-7e. There are two spherical radii SR1 and SR2 machined into the end surface 22 used for molding. The process of making the pin includes machining the two radii using end mills, then polishing by hand the two surfaces which results in some blending in between the two spherical radii. This surface directly contacts the liquid silicone and is used to shape (mold) the final optical center surface on the optic.

The molding process uses “compression molding”. The liquid silicone is poured into the mold, the two halves of the mold are put together, and the mold is then placed in a heated press under approximately a ton of force for a period of time. After the period is up, the press opens and the mold is removed and allowed to cool prior to removing the molded part. This is the same process used with applicant's standard IOL's.

The material used has a small shrinkage factor when transforming from the liquid to the solid state. This shrinkage and the tumbling process results in spherical radii that are different from that of the pins.

After molding, the IOL's are tumbled in an Alox (aluminum oxide), glass beads (of 3.0 and 0.75 mm diameter) and isopropyl alcohol mixture for a period of time. One lot (<100 IOL's) is tumbled together in a small glass jar on a industrial “rock tumbler”, manufactured by Topline. Here too, the process is the same as used with applicant's standard IOL's.

The tumbling process has two effects: first it removes flash and rounds off sharp edges, and second, it is believed that the alcohol swells the silicone, allowing unbound silicone molecules to be flushed out. This results in an additional change in the spherical radii at 3 which gets us to the final product.

The lens shrank resulting in an absence of discrete radii SR1-SR5, and thus ends up not a multiple power lens after implantation into the eye. The resulting blended design after completion does not cause separate images on wavefront analysis after implantation into an eye, as does a multifocal lens, but actually provides a central defocus curve which provides additional focusing power and actually results in an extended region of depth of field about the far point of the patient's vision. Thus, a desired depth of field increase about the focal point occurs, and the retinal image range has been determined to be superior than that of a standard accommodating intraocular lens or other intraocular lenses. Thus, the lens functions by extending the range of accommodation about the far point by increasing the static depth of field. A patient's vision is improved by both accommodation of the lens by axial movement and arching of the optic and by virtue of an increased depth of field.

The attached waveforms of Exhibit 1 and Exhibit 2 illustrate differences between applicant's standard accommodating intraocular lens AT-45 and the present lens with the central area as described. Exhibit 1 illustrates wavefront verification display for the AT-45, and shows a relatively small focus area in the retinal spot pattern in the lower left hand corner of Exhibit 1. Exhibit 2 is a wavefront verification display for the present lens and it can be seen in the retinal spot pattern at the lower left display in Exhibit 2 that the present lens provides a single point focus in the eye.

Accordingly, there has been shown and described a lens that can comprise a silicone optic and silicone flat solid haptic plates, loops that can be of a different material than the plate or the same, and a fixation centration device at the end of each loop allowing for movement of the plate haptics and loops along the tunnels formed in the fusion of the anterior and posterior capsules of the human capsular bag, and wherein the anterior surface of the optic has a central area of increased power of 1 diopter or less. The lens can be implanted in the non-dominant or dominant eye.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

1. A method of manufacturing an intraocular lens with a flexible solid optic having a central area of increased power of one diopter or less on one side to enable an extended depth of field about the far point of a patient's vision, to give the eye, after implantation, a single focal point when evaluated by wavefront analysis comprising
molding the optic of a transparent plastic material using an optical pin designed to give the optic on one side a higher central power, or more than the power measured after manufacture, than the power in the periphery of the lens, and
processing the molded optic to integrate the radii to cause the central area of the optic to be reduced to cause the lens optic to produce a single focal point on wavefront analysis after implantation in the eye.

2. A method as in claim 1 wherein the optic has two or more radially extending portions from the optic having centration fixation structures at their distal ends such that the optic of the lens can move anteriorly with contraction of the ciliary muscle of the eye.

3. A method as in claim 1 wherein the slurry includes aluminum oxide and alcohol.

4. An intraocular lens manufactured according to the method of claim 1.

5. An accommodating intraocular lens manufactured according to the method of claim 1.

6. A method as in claim 1 wherein the pin gives a central power of 1.5 diopters or more.

7. A method as in claim 1 wherein the resulting central area is reduced to 1.0 diopter or less.

8. A method for improving near vision of an eye of a patient comprising the steps of implanting in the eye of the patient an intraocular lens which has a flexible lens body having normally anterior and posterior sides and including a flexible solid optic, the optic having a central area of increased power of 1 diopter or less to enable an extended region of depth of field about the far point of a patient's vision to give the patient, after implantation, a single focal point when evaluated by wavefront analysis, the lens body having two or more extended portions from the optic such that the lens can move anteriorly and posteriorly with contraction and relaxation of the ciliary muscle of the eye, and the lens being sized to be implanted into the capsular bag of the eye such that contraction of the ciliary muscle can cause the optic of the lens within the capsular bag behind the iris to move forward toward the iris with its contraction, the lens being formed with a central power of 1.5 diopters more than the power in the periphery of the lens on one side, and tumbled in a slurry of glass beads to integrate the radii on that side and provide the central area that provides a single focal point on wavefront analysis after implantation in the eye.

9. A method as in claim 8 wherein after tumbling, the central area is 1.0 diopter or less.

10. A method as in claim 8 wherein the lens is an accommodating intraocular lens.

11. An intraocular lens for implantation into the eye of a patient comprising a flexible lens body having normally anterior and posterior sides and including a flexible solid optic,
the lens body having two or more radially extending portions from the optic having centration fixation structures at their distal ends such that the optic of the lens can be implanted in the capsular bag of the eye and move anteriorly and posteriorly with contraction and relaxation of the ciliary muscle of the eye,
the optic having a central area of increased power of 1 diopter or less on one side to enable an extended depth of field about the far point of a patient's vision to give the patient, after implantation, a single focal point when evaluated by wavefront analysis,
the lens being sized to be implanted into the capsular bag of the eye such that contraction of the ciliary muscle can cause the optic of the lens within the capsular bag behind the iris to move forward toward the iris, and
wherein the lens is formed with a central power 1.5 diopters or more than the power in the periphery of the lens on the one side, and the lens is tumbled with a slurry of glass beads to integrate the radii on that side and provide the central area to provide a single focal point on wavefront analysis after implantation in the eye.

12. An accommodating intraocular lens for implantation into the eyes of a patient comprising a flexible lens body having normally anterior and posterior sides, including a flexible solid optic,
the lens body having two or more radially extending portions from the optic having centration fixation structures at their distal ends such that the optic of the lens can move anteriorly with contraction of the ciliary muscles of the eye,
the optic having a central area of increased power of 1 diopter or less on one side to enable an extended depth of field about the far point of a patient's vision to give the patient, after implantation, a single focal point when evaluated by wavefront analysis and,
the lens being sized to be implanted into the capsular bag of the eye such that contraction of the ciliary muscle causes the optic of the lens within the capsular bag behind the iris to move forward toward the iris.

13. An accommodating lens according to claim 12 wherein the extending portions are plate haptics.

14. An accommodating lens according to claim 12 wherein the extending portions are plate haptics with a narrowing of the plate junctions adjacent to the optic.

15. An accommodating lens according to claim 12 wherein the extending portions are plate haptics with a widening of the plate junctions adjacent to the optic.

16. An accommodating lens according to claim 13 wherein the plate haptics are flat and solid and have parallel sides.

17. An accommodating lens according to claim 13 wherein the plate haptics are flat and solid and have one or more grooves across their flat sides.

18. An accommodating lens according to claim 13 wherein the plates are flexible throughout their length.

19. An accommodating lens according to claim 12 wherein the optic may deform when subjected to posterior vitreous pressure, resulting in accommodative arching.

20. A method for improving near vision of a non-dominant eye of a patient comprising the steps of
implanting in the non-dominant eye of the patient an accommodating intraocular lens which has a flexible lens body having normally anterior and posterior sides and including a flexible solid optic, the optic having a central area of increased power of 1 to enable an extended region of depth of field about the far point of a patient's vision to give the patient, after implantation, a single focal point when evaluated by wavefront analysis, the lens body having two or more extending portions from the optic such that the lens can move anteriorly and posteriorly with contraction and relaxation of the ciliary muscle of the eye, and the lens being sized to be implanted into the capsular bag of the eye such that contraction of the ciliary muscle can cause the optic of the lens within the capsular bag behind the iris to move forward toward the iris with its contraction.

21. A method as in claim 20 comprising the further steps of
implanting in the dominant eye of the patient an accommodating intraocular lens which has a flexible lens body having normally anterior and posterior sides and including a flexible solid optic, the lens body having two or more radially extending portions from the optic such that the optic of the lens can move anteriorly with contraction of the ciliary muscle of the eye.

22. A method of manufacturing an intraocular lens with a flexible solid optic having a central area of increased power of one diopter or less on one side to enable an extended depth of field about the far point of a patient's vision, to give the eye, after implantation, a single focal point when evaluated by wavefront analysis comprising
molding the optic of a transparent plastic material using an optical pin designed to give the optic on one side a higher central power than the power measured after manufacture than the power in the periphery of the lens, and
tumbling the thus molded optic with a slurry of glass beads to remove any flashing, smooth the edges and integrate the radii to cause the central area of the optic to be reduced to cause the lens optic to generate a single focal point on wavefront analysis after implantation in the eye.
